(19) United States
(12) Patent Application Publication
(10) Pub. No.: US 2010/0216168 A1
(43) Pub. Date: Aug. 26, 2010

(54) METHODS FOR EVALUATING ANGIOGENIC POTENTIAL IN CULTURE

(75) Inventors: Jamie Heinzman, Pittsburgh, PA (US); Stacey Brower, Pittsburgh, PA (US); Jason Bush, Pittsburgh, PA (US); Zhibao Mi, Pittsburgh, PA (US)

Correspondence Address: COOLEY LLP;ATTN: Patent Group, Suite 1100, 777 - 6th Street, NW, WASHINGTON, DC, 20001, US

(73) Assignee: PRECISION THERAPEUTICS, INC., Pitrsburgh, PA (US)

(21) Appl. No.: 12/530,541

(22) PCT Filed: Mar. 24, 2008
(86) PCT No.: PCT/US2008/058001

§ 371 Date: Mar. 3, 2010

Related U.S. Application Data
(60) Provisional application No. 60/907,181, filed on Mar. 23, 2007.
(60) Provisional application No. 61/029,164, filed on Feb. 15, 2008.

(51) Int. Cl. G01N 33/574

(52) U.S. Cl. 435/7.23

(57) ABSTRACT

The present invention provides a method of evaluating the angiogenic potential of a tumor, and for predicting the efficacy of anti-angiogenic therapies on an individualized basis. The method of the invention involves preparing an angiogenic signature for malignant cells in culture by assaying for the presence or level of one or more angiogenesis-related factors selected from VEGF/VPF, IL8/CXCL8, TGF-β1, TGF-β2, TGF-β3, bFGF/FGF-2, EGF, PDGF-AA, PDGF-AA/BB, IP-10, and Flt-3 ligand. The angiogenic signature may be prepared from cultures maintained under normoxic and/or hypoxic environments. The invention may be used in conjunction with chemoresponse testing of anti-tumor agents, to predict or suggest a combination therapy for cancer patients.

### PRIORITY

This application claims priority to U.S. Provisional Application Ser. No. 60/907,181, filed Mar. 23, 2007, and to U.S. Provisional Application Ser. No. 61/029,164, filed Feb. 15, 2008. The contents of U.S. Provisional Application Ser. No. 60/907,181 and U.S. Provisional Application Ser. No. 61/029,164 are incorporated herein by reference in their entirety.

### FIELD OF THE INVENTION

The present invention relates generally to methods for evaluating the angiogenic potential of a tumor in vitro, and for predicting efficacy of anti-angiogenic therapy for cancer patients on an individualized basis.

### BACKGROUND OF THE INVENTION

Tumor angiogenesis depends on a balance between a complex assortment of activating and inhibiting factors that are secreted by tumor cells as well as non-malignant cells including macrophages and fibroblasts, which may infiltrate the tumor. As a tumor grows, the existing blood supply becomes inefficient at supporting the tissue and areas of the tumor become hypoxic. The hypoxic condition triggers the tumor to enhance the expression of angiogenic factors, triggering the formulation of new blood vessels to support the growing tissue (Pilch et al., 2001, Int. J. Gynecol. Cancer 11: 137-142; Kuroki et al., 1996, J. Clin. Invest. 98(7): 1667-1675). Angiogenesis is required for tumor survival as well as further growth, progression, and metastasis (Mukherjee et al., 2002, Brit. J. of Cancer 92: 350-358). In fact, high tumor vascular density is correlated with negative patient outcomes, including shorter progression-free interval and reduced overall survival (Pilch et al., 2001; Muller et al., 1997, Proc. Natl. Acad. Sci. 94: 7192-7197; Mohammed et al., 2007, Brit. J. of Cancer 96: 1092-1100).

Angiogenesis is a highly regulated process. Recruitment of resting vascular endothelial cells (“VEC”) in response to the increased metabolic demands of a growing tumor mass follows stable pathways that are normally invoked in wound healing, reproductive physiology, and in ontogeny (Sen et al., 2002, Am. J. Physiol. Heart Circ. Physiol. 282: H1821-7). To stimulate angiogenesis, tumors may upregulate their production of a variety of angiogenic factors, including the fibroblast growth factors (FGF and bFGF) (Kandel et al., 1991) and vascular endothelial cell growth factor/vascular permeability factor (VEGF/VPF). However, malignant tumors may also generate inhibitors of angiogenesis, including angiostatin and thrombospondin (Chen et al., 1995; Good et al., 1990; O'Reilly et al., 1994). The angiogenic phenotype may result from the balance of positive and negative regulators of neovascularization (Good et al., 1990; O'Reilly et al., 1994; Parangi et al., 1996; Rastinejad et al., 1989). In diseased tissue, this balance may shift in favor of the positive regulators (Terman et al., 2000, Einstein Quart. J. Biol. and Med. 18:59-66). Several other endogenous inhibitors of angiogenesis have been identified, although not all are associated with the presence of a tumor. Other endogenous inhibitors include, platelet factor 4 (Gupta et al., 1995; Maione et al., 1990), interferon-alpha, interferon-inducible protein 10 (Angiolillo et al., 1995; Strieter et al., 1995), which is induced by interleukin-12 and/or interferon-gamma (Voest et al., 1995), gro-beta (Cao et al., 1995), and the 16 kDa N-terminal fragment of prolactin (Clapp et al., 1993).

Hypoxic conditions can trigger a tumor to enhance the expression of angiogenic factors in vivo. For example, VEGF is secreted by cancer cells as well as supporting stomal cells, including fibroblasts, especially during conditions of hypoxia (Pilch et al., 2001). Further, in vitro studies have shown that stromal cells cultured in hypoxic growth conditions secrete higher levels of critical angiogenesis-inducing factors than cells cultured in normoxic conditions (Mukherjee et al., 2002). High expression of VEGF is observed in many tumor types and is correlated with aggressive tumor growth and metastasis (Shi et al., 2007, Pathology 39(4): 396-400; Yang et al., 2003, The New England J. of Med. 349(5): 427-434; Mohammed et al., 2007).

Regulation of VEGF expression is complex, occurring at both the transcription and translation stages of protein synthesis, with many ligand-receptor interactions (Mukherjee et al., 2002; Kuroki et al., 1996; Wang et al., 2004, Angiogenesis 7: 335-345). Expression of VEGF is up-regulated by hypoxia inducible factor-1 (HIF-1), which binds to the VEGF promoter, increasing transcription of VEGF (Hicklin et al., 2005, J. Clin. Onc. 23(5): 1011-1027). Once expressed, VEGF has the ability to bind to two endothelial cell-specific receptors, kinase domain receptor (KDR) and fms-like tyrosine kinase (Flt-1) to initiate angiogenesis among other survival signals (Kim et al., 1993, Nature 362: 841-844; Muller et al., 1997). In addition to changes in endothelial cells, VEGF increases vasculature permeability, earning its other name as vascular permeability factor (VPF). The vascular leakage allows proteins, such as matrix metalloproteases (MMPs), to be deposited in the extracellular fluid. MMPs break down the extracellular matrix and allow endothelial cells to migrate and invade areas in close proximity to the tumor (Wang et al., 2004; Hicklin et al., 2005).

The roles of several angiogenesis factors are summarized in Table 1, below. For example, some factors function by mediating VEGF production, such as basic Fibroblast Growth Factor (bFGF/FGF-2) and Epidermal Growth Factor (EGF). Others factors function by modifying the extracellular environment of the tumor, including bFGF, Interleukin-8 (IL-8/CXCL8), and Platelet-derived Growth Factors-AA and -AA/BB (PDGFs). Induction of endothelial cell growth is accomplished by IL-8, Fms Related Tyrosine Kinase (Flt-3 Ligand), and PDGFs, while EGF and Transforming Growth Factors-β1, β2, and β3 (TGFs) are involved in tumor growth and proliferation. Lastly, IP-10/CXCL10 inhibits tumor and endothelial cell growth and is inversely correlated with VEGF production.

TABLE 1
Description and role of angiogenesis-related factors.

| Angiogenesis-Related | |
|---|---|
| Factor | Role in Angiogenesis |
| Vascular Endothelial | Signaling protein for angiogenesis that works by binding, |
| Growth Factor/Vascular | dimerizing, and phosphorylating external tyrosine kinase |
| Permeability Factor | receptors. Can be induced by hypoxia through the release of |
| (VEGF/VPF) | Hypoxia Inducible Factor (HIF) (Muller et al., 1997; Shi et al., |
| | 2007; Wang et al., 2004). |
| Basic Fibroblast Growth | Stimulates production of basement membranes via formation |
| Factor | of extracellular matrix. Aids in angiogenesis in tumors by |
| (bFGF/FGF-2) | mediating VEGF production (Shi et al., 2007, Kim et al., |
| | 1993). |
| Interleukin-8 | A chemokine that regulates angiogenesis by promoting |
| (IL-8/CXCL8) | survival of endothelial cells, stimulating matrix |
| | metalloproteinases, and increasing endothelial permeability |
| | (Cheng et al., 2008, Cytokine 41(1): 9-15; Petreaca et al., |
| | 2007, Mol. Biol. Cell. 18(12): 5014-5023). |
| Epidermal Growth Factor | Factor commonly expressed in carcinomas involved in tumor |
| (EGF) | growth, proliferation, and differentiation by stimulation of |
| | intrinsic protein-tyrosine kinase activity, resulting in DNA |
| | synthesis. Also, induces VEGF, IL-8, and bFGF release by |
| | tumor cells (De Luca et al., 2008, J. Cell. Physiol. 214(3): 559-67; |
| | Hicklin et al., 2005). |
| Fms-related Tyrosine | Cytokine that assists in proliferation and maturation of |
| Kinase | hematopoietic progenitor cells (Harada et al., 2007, Int. J. |
| (Flt-3 Ligand) | Oncol. 30(6): 1461-8). |
| Platelet-derived Growth | Mitogenic factors for fibroblasts, smooth muscle, and |
| Factors | connective tissue that can be induced by VEGF and bFGF. |
| (PDGF-AA, -AA/BB) | Induce endothelial cell survival by recruiting stromal cells for |
| | VEGF production (Reinmuth et al., 2007, Int. J. Oncol. 31(3): |
| | 621-626; Hicklin et al., 2005). |
| Interferon-gamma-inducible | Inhibits tumor growth by regulating lymphocyte chemotaxis |
| Protein 10 | and inhibiting endothelial cell growth. Down-regulation |
| (IP-10) | correlated with poor prognosis. Reverse-correlated with |
| | VEGF (Sato et al., 2007, Br. J. Cancer 96(11): 1735-1739). |
| Transforming Growth | Cytokines that control several biological processes including |
| Factors | cell growth, proliferation, differentiation, and apoptosis. |
| (TGF-β1,2,3) | Pathological conditions such as cancer are can be linked to |
| | modifications of these growth factors (Mourskaia et al., 2007, |
| | Anticancer Agents Med. Chem. 7(5): 504-514). |

Several strategies have been developed for targeting angiogenesis, such as monoclonal antibodies against VEGF (e.g., Bevacizumab), soluble VEGF receptors (e.g., VEGF Trap), tyrosine kinase receptor inhibitors (e.g., inhibitors of VEGFR 1, 2, and/or 3, FLT3, PDGFR-α and/or β), inhibitors of endothelial cell proliferation (e.g., Endostatin, Angiostatin, Thalidomide), inhibitors of extracellular matrix breakdown (e.g., Marimastat, Neovstat), and inhibitors of vascular adhesion (e.g., Vitaxin). However, there is a need for methods that aid individualized treatment decisions with respect to the emerging array of anti-angiogenic agents, such as in vitro methods that accurately evaluate the angiogenic potential of a patient's tumor, as well as methods that predict the efficacy of an anti-angiogenic therapy for a particular patient.

### SUMMARY OF THE INVENTION

The present invention provides methods for evaluating, in vitro, the angiogenic potential of a tumor in vivo, and provides methods for predicting the efficacy of anti-angiogenic therapy on an individualized basis.

In one aspect, the invention provides a method for creating an angiogenic signature for a tumor specimen. The method comprises culturing malignant (e.g., tumor) cells from a patient specimen, and testing the cell culture for the presence and/or levels of angiogenesis-related factors. The angiogenesis-related factors may be selected from VEGF/VPF, bFGF/FGF-2, IL-8/CXCL8, EGF, Flt-3 ligand, PDGF-AA, PDGF-AA/BB, IP-10/CXCL10, TGF-β1, TGF-β2, TGF-β3, VEGFR, HIF1-alpha, EGFR, HER-2, TGF-alpha, TNF-alpha, thrombospondin, and angiogenin. The angiogenic signature allows for the evaluation of the tumor's angiogenic potential in vivo, including, in some embodiments, evaluation of the aggressiveness of the tumor and potential for metastasis. In some embodiments of the invention, a tumor specimen (e.g., biopsy) is cultured so as to enrich for malignant cells. In these and other embodiments, the cultures may be maintained in a normoxic environment, or alternatively, hypoxic and normoxic cultures may be established in sequence or in parallel such that the presence and/or levels of angiogenesis-related factors may be assayed under both conditions.

In a second aspect, the invention provides a method for preparing a predictive model that finds use in predicting angiogenic and/or metastatic potential of a tumor, as well as predicting efficacy of anti-angiogenic therapy. In accordance with this aspect, the method comprises culturing malignant cells from a plurality of patient tumor specimens, and preparing an angiogenic signature, as described herein, for each tumor specimen. The angiogenic signatures are matched with anti-angiogenic treatment regimens and clinical outcomes for the patients from which the specimens originated. Together, the information creates a predictive model for evaluating angiogenic potential, and for predicting the efficacy of anti-angiogenic therapy in connection with further tumor specimens.

In a third aspect, the invention provides a method for predicting the efficacy of an anti-angiogenic agent for a cancer patient. In accordance with this aspect, the method comprises culturing malignant cells (e.g., tumor cells) from a patient specimen, and preparing an angiogenic signature as described herein. The angiogenic signature may be evaluated with respect to numbers and levels of positive and negative regulators of angiogenesis secreted by the tumor, or alternatively, may be matched with a model signature (e.g., predictive model as described herein) to correlate the angiogenic signature to an appropriate angiogenic treatment regimen and patient prognosis. In certain embodiments, the method further comprises chemoresponse testing of traditional cancer agents, so that an effective combined therapy may be selected on an individualized basis. The angiogenic signature as described herein may be conveniently prepared in conjunction with conventional cell culture methods used for chemoresponse testing.

### BRIEF DESCRIPTION OF THE DRAWINGS

FIG. 1. shows that culture growth is comparable under normoxic and hypoxic conditions. A linear regression of the normoxic versus hypoxic percent confluency of each of 50 samples shows the confluencies to be similar within a given sample. Many samples reached 100% confluency in both conditions, so less than 50 points appear on the graph.

FIG. 2. shows the linear correlations between normoxic and hypoxic conditions for VEGF expression. Fifty cell sources (45 primary tumor cultures and 5 immortalized cell lines) were evaluated for VEGF expression measured by ELISA-based assay. The larger graph divides the specimens by tumor type, while the inset combines all data.

FIG. 3. shows the differential expression of angiogenesis-related factors across samples. Differential levels of expression are evident across all patients for the angiogenesis-related factors tested. Correlation coefficients indicate that the differences in VEGF are not correlated to differences in expression of the other angiogenesis-related proteins.

### DETAILED DESCRIPTION

The present invention provides methods that allow for evaluating the angiogenic and/or metastatic potential of a tumor in vitro by preparing angiogenic signatures for cultured tumor cells.

Angiogenic Signature

The present invention provides a method for evaluating the angiogenic potential of tumor cells. The invention comprises culturing malignant cells from a patient tumor specimen, and determining the presence or level, in culture, of one or more angiogenesis-related factors selected from VEGF/VPF (e.g., VEGF-A), IL8/CXCL8, TGF-β1, TGF-β2, TGF-β3, bFGF/FGF-2, EGF, PDGF-AA, PDGF-AA/BB, IP-10, and Flt-3 ligand (see Table 1). Generally, the angiogenic signature includes values for at least three, four, five, six, seven, eight, nine, ten, or all of these angiogenesis-related factors. In many embodiments, the angiogenic signature comprises the level of VEGF expression (e.g., secretion) from the cultured cells, in combination with the level of at least one or two additional positive regulators of angiogenesis, such as EGF and IL-8. The angiogenic signature may be prepared by testing for the presence and/or levels (e.g., concentration) of the angiogenesis-related factors secreted into cell culture media by the cultured cells using, for example, standard immunological-based assays (e.g., ELISA), as described herein. The levels of the angiogenesis-related factors may be compared to levels determined for one or more control cell cultures, or for one or more control factors that are not related to angiogenesis. Such factors are known in the art. The angiogenic signature may be a quantitative or semi-quantitative measurement.

The angiogenic signature may further comprise a determination of the presence and/or level of additional angiogenesis-related factors, or factors related to tumor aggressiveness or metastasis. Such additional factors may be secreted from cultured cells or may be cell-surface markers. For example, the angiogenic signature may further comprise a determination of the presence and/or level of one or more of VEGFR, HIF1-alpha, EGFR, HER-2, TGF-alpha, TNF-alpha, thrombospondin, and angiogenin. In certain embodiments, the method of the invention tests for several positive regulators or markers of angiogenesis (e.g., two, three, or four), and optionally, one or more (e.g., two) negative regulator(s) or markers of angiogenesis.

The angiogenic signature is determined for cultured malignant cells (e.g., tumor cells). The tumor cells may be obtained via a biopsy specimen from a cancer patient in need of treatment. The tumor may be from a solid tumor, or may be soft-tissue tumor cells, metastatic tumor cells, leukemic tumor cells, and/or a lymphoid tumor cell. Exemplary cancers include lung, breast, and colon cancers. However, the invention finds use in a variety of malignancies, including ACTH-producing tumors, acute lymphocytic leukemia, acute nonlymphocytic leukemia, cancer of the adrenal cortex, bladder cancer, brain cancer, cervix cancer, chronic lymphocytic leukemia, chronic myelocytic leukemia, colorectal cancer, cutaneous T-cell lymphoma, endometrial cancer, esophageal cancer, Ewing's sarcoma, gallbladder cancer, hairy cell leukemia, head and neck cancer, Hodgkin's lymphoma, kidney cancer, liver cancer, malignant peritoneal effusion, malignant pleural effusion, melanoma, mesothelioma, multiple myeloma, neuroblastoma, non-Hodgkin's lymphoma, osteosarcoma, prostate cancer, retinoblastoma, soft-tissue sarcoma, squamous cell carcinomas, stomach cancer, testicular cancer, thyroid cancer, trophoblastic neoplasms, vaginal cancer, cancer of the vulva, Wilm's tumor. Other cancers include a fibrosarcoma, myosarcoma, liposarcoma, chondrosarcoma, osteogenic sarcoma, chordoma, angiosarcoma, endotheliosarcoma, lymphangiosarcoma, lymphangio-endotheliosarcoma, synovioma, mesothelioma, leiomyosarcoma or rhabdomyosarcoma, epithelial carcinoma, glioma, astrocytoma, medullobastoma, craniopharyngioma, ependymoma, pinealoma, hemangio-blastoma, acoustic neuroma, oligodendroglioma, meningioma, melanoma, neurobastoma, retinoblastoma, leukemia, and lymphoma.

In accordance with the invention, the cell culture may be maintained under normoxic conditions. As disclosed herein, while hypoxic conditions may result in somewhat higher or lower levels of some angiogenesis-related factors, the levels of many angiogenesis-related factors secreted from cultured tumor cells are similar or linear between normoxic and hypoxic conditions, allowing for the presence or levels to be tested in either or both conditions (e.g., normoxic and/or hypoxic). For example, as described herein, the levels of VEGF, bFGF, IL-8, EGF, TGF-β2, PDGF-AA, PDGF-AA/BB, and IP-10 secreted from cultured tumor cells are similar or linear between normoxic and hypoxic environments (see Table 2). In certain embodiments of the invention, cell cultures are maintained in normoxic and hypoxic environments, either in sequence or in parallel, and the presence and/or levels of the angiogenesis-related factors are tested under both conditions. A hypoxic condition or environment may be, for instance, about 0.5% to about 15% oxygen, such as from about 1% to about 5% oxygen. Normoxic conditions include conditions at about 18% to about 23% oxygen, such as about 21%.

In certain embodiments, the cultured tumor cells may be from a lung or breast cancer specimen. As shown herein, the levels of secreted angiogenesis-related factors are similar or linear under normoxic and hypoxic environments for lung and breast cancer specimens, and thus, such cells may be cultured under either or both conditions with the resulting angiogenic signature reasonably representing the profile secreted under the in vivo hypoxic environment.

The levels of the angiogenesis-related factors may be compared to one or more controls. For instance, a control may be the level(s) of the particular angiogenesis-related factors secreted from cultured cells derived from a patient known to be responsive, or not responsive, to a particular anti-angiogenic therapy, or derived from a patient having a particular disease progression or angiogenic phenotype. For example, the level of VEGFNPF, IL8/CXCL8, TGF-β1, TGF-β2, TGF-β3, bFGF/FGF-2, EGF, PDGF-AA, PDGF-AA/BB, IP-10, and Flt-3 ligand, VEGFR, HIF1-alpha, EGFR, HER-2, TGF-alpha, TNF-alpha, thrombospondin, and angiogenin may be the same, higher or lower when compared to the levels of the same marker from a control tumor specimen. Differences in the levels of these markers are generally significant where the differences are at least about 1.5 fold, but may be 50 fold or more. Additional controls include the level of one or more secreted markers that are not related to angiogenesis, and which are preferably secreted in similar or equal amounts under normoxic and hypoxic environments in vitro.

The angiogenic signature may be used to evaluate the angiogenic potential of the tumor in vivo, and to select an appropriate anti-angiogenic therapy. For example, where VEGF is the substantial (or most significant) positive regulator of angiogenesis secreted from the cultured cells, a VEGF inhibitor such as Bevacizumab or VEGF trap might be an appropriate therapy for the patient, that is, to directly target VEGF in vivo. In contrast, where multiple positive regulators of angiogenesis (e.g., including those that do not substantially relate to regulating VEGF expression) are expressed at significant or substantial levels by the cultured cells (suggesting potential evasion by the tumor of a single targeted angiogenesis regulator), inhibition of multiple angiogenic regulators, or downstream events, might be more appropriate. Such might include inhibition of tyrosine kinase receptor(s), including receptors for VEGF, bFGF, and PD-ECGF, or therapy to inhibit the proliferation or survival of endothelial cells. Alternatively, where several positive regulators of angiogenesis are secreted at substantial or significant levels from the cultured cells, therapy to inhibit vascular cellular adhesion or to inhibit degradation of the extracellular matrix might also be desirable. Further, combination anti-angiogenic therapy may be warranted in such cases.

Therefore, the present invention enables the prediction of therapeutic efficacy with several available anti-angiogenic strategies, such as (but not limited to) monoclonal antibodies against VEGF (e.g., bevacizumab), soluble VEGF receptors (e.g., VEGF Trap), tyrosine kinase receptor inhibitors (e.g., inhibitors of VEGFR 1, 2, and/or 3, FLT3, PDGFR-α and/or β), inhibitors of endothelial proliferation (e.g., Endostatin, Angiostatin, Thalidomide), inhibitors of extracellular matrix breakdown (e.g., Marimastat, Neovstat), and inhibitors of vascular adhesion (e.g., Vitaxin).

In particular, Bevacizumab (Avastin®, Genentech) is a recombinant humanized monoclonal antibody, approved for cancer treatment by the FDA in 2004 (Ignoffo et al., 2004, Am. J. Health-Syst. Pharm. 61: S21-S26). This drug binds VEGF with high specificity, neutralizing the growth factor and preventing the interaction of VEGF with its receptors. Therefore, proliferation of endothelial cells is inhibited (Kim et al., 1993; Wang et al., 2004). While bevacizumab has a high affinity for all VEGF isoforms, the drug does not bind other related growth factors such as EGF, bFGF, or PDGFs (Ignoffo et al., 2004). Because of both the specificity of this antibody for VEGF and the significant biological implications, bevacizumab has been approved for the treatment of: primary and metastatic colorectal cancer; non-small cell lung cancer; and metastatic breast cancer (Her2−, no prior chemotherapy, and in combination with paclitaxel).

Culturing Malignant Cells

In accordance with the invention, malignant cells from a patient specimen are cultured for establishing an angiogenic signature. The cell culture may be such that the culture is enriched for malignant cells. For example, the cell culture may be grown from cohesive multicellular particulate(s) of the tumor tissue, in contrast to dissociated or suspended cells. By maintaining the abnormal proliferating cells within cohesive multicellular particulate(s) of the originating tissue for initial tissue culture monolayer growth, rather than dissociating or suspending the abnormal proliferating cells, growth of the abnormal proliferating cells is facilitated versus the overgrowth of fibroblasts or other cells. Establishing cell cultures from cohesive multicellular particulates may preserve the profile of secreted factors and cell surface markers. While angiogenesis is a dynamic process influenced by a variety of factors produced by a variety of cell types in vivo, the angiogenic signature produced by tumor cells enriched and grown in culture, as described herein, provides for a meaningful evaluation of the tumors potential in vivo.

According to these embodiments, the tumor cells are prepared by first separating a tissue specimen from the patient into multicellular particulates in a mechanical fashion. In an exemplary embodiment, a tumor biopsy of at least 17 mg of non-necrotic, non-contaminated tissue sample is harvested from the patient by any suitable biopsy or surgical procedure and is typically placed in a shipping container for transfer to a laboratory to culture the cells. A specimen can be taken from a patient at any relevant site including, but not limited to, tissue, ascites or effusion fluid. Samples may also be taken from body fluid or exudates as is appropriate. The tissue sample is then minced with sterile scissors. A portion of the minced sample may be reserved, snap-frozen and preserved for additional analysis, such as genomic analysis. Using sterile forceps, each undivided tissue sample quarter is then placed in 3 ml sterile growth medium (containing 0-20% calf serum and a standard amount of penicillin and streptomycin) and systematically minced by using two sterile scalpels in a scissor-like motion or a mechanically equivalent manual or automated device having opposing incisor blades. This cross-cutting motion creates smooth cut edges on the resulting tumor multicellular particulates. The tumor particulates may have a size of about 1 mm³, but the tissue specimen may be mechanically separated into multicellular particulates measuring, for example, from about 0.25 to about 1.5 mm³.

When culturing certain cell types, such as ovarian and colorectal tumor tissue, it may be desirable to treat the multicellular particulates with a Collagenase II and DNase cocktail to further reduce the size of the multicellular particulates prior to culturing. For instance, the multicellular particulates may be treated with a cocktail of about 0.025% Collagenase and about 0.001% DNase.

In some embodiments, the particulates are agitated to substantially release tumor cells from the tumor explant particles. Such agitation includes any mechanical means that enable the enhanced plating of tumor cells and includes, but is not limited to, shaking, swirling, or rapidly disturbing the explant particles. These procedures may be done by hand, for instance, by sharply hitting the container against a solid object or by the use of mechanical agitation. For instance, a standard vortex mixer may be used. This agitation step typically increases the number of adherent tumor cells, as compared to non-agitated replicate samples after about 12-48 hours or more of incubation. Chemicals or enzymes may be employed to facilitate the release of tumor cells from the tumor explant. Enzymatic agitation with enzymes may include collagenase, DNase or dispase.

In some embodiments, following initial culturing of the multicellular tissue explant, the tissue explant is removed from the growth medium at a predetermined time as described in US Published Application No. 2007/0059821, which is hereby incorporated by reference in its entirety. Generally, the explant is removed from the growth medium prior to the emergence of a substantial number of stromal cells from the explant. The explant may be removed according to the percent confluency of the cell culture. For example, the explant may be removed at about 10 to about 50 percent confluency. In a preferred embodiment, the explant is removed at about 15 to about 25 percent confluency, such as at about 20 percent confluency. By removing the explant in this manner, a cell culture monolayer predominantly composed of malignant cells (e.g., tumor cells) is produced. In turn, a substantial number of normal cells, such as fibroblasts or other stromal cells, fail to grow within the culture. Ultimately, this method of culturing a multicellular tissue explant and subsequently removing the explant at a predetermined time allows for increased efficiency in both the preparation of cell cultures and subsequent assays.

Multicellular particulates are grown to form a tissue culture monolayer. Growth of the cells is monitored by counting the cells in the monolayer on a periodic basis, without killing or staining the cells, and without removing any cells from the culture flask. The cells may be counted visually or by automated methods, either with or without the use of estimating techniques known in the art. For example, the cells in a representative grid area may be counted and multiplied by the number of grid areas. Data from periodic counting may then be used to determine growth rates, which may or may not be considered to parallel growth rates of the tumor cells in vivo.

Protocols for monolayer growth rate generally use a phase-contrast inverted microscope to examine culture flasks incubated in a 37° C. (5% CO₂) incubator. When the flask is placed under the phase-contrast inverted microscope, ten fields (areas on a grid inherent to the flask) are examined using the 10× objective. The ten fields should be non-contiguous, or significantly removed from one another, so that the ten fields are a representative sampling of the entire flask. A percentage of cell occupancy for each examined field is noted, and these percentages are averaged to provide an estimate of the percent confluency in the cell culture. When patient samples have been divided between two or among three or more flasks, an average cell count for the total patient sample should be calculated. The calculated average percent confluency is entered into a process log to enable compilation of data and plotting of growth curves over time.

The applicable formula is: percent confluency=estimate of the area occupied by cells/total area of the in an observed field.

Monolayer cultures may be photographed to document cell morphology and culture growth patterns.

The growth rate of the cells may be determined. The growth may also be monitored by observing the percent of confluency of the cells in a flask. These data provide information valuable as a correlation to possible growth of the tumor in the patient, as well as for the interpretation of the results of a chemosensitivity assay (or “chemoresponse assay”), if conducted. The percent of confluency of the cultured cells is plotted as a function of time after the initial seeding of the tissue specimen:

Slow growth rate: 25% confluent after 19 days

Moderate growth rate: 60% confluent after 21 days

Fast growth rate: 90% confluent after 21 days

To assay for secreted factors (e.g., angiogenesis-related factor), about 5,000 to about 50,000 cells, such as about 20,000 cells, from the cell culture may be seeded in an appropriate volume of media, such as 0.5 mL or 1.0 mL media. Cells are allowed to incubate undisturbed for two to five days (e.g., about 96 hours), under desired culture conditions (which may include 37° C. and 5% CO₂). At the conclusion of the incubation period, cell culture media is aspirated from each well using a sterile pipette, transferred and split into separate cryovials, and stored frozen at −80° C. until the time of assay.

Assay Methodologies

The presence or level of angiogenesis-related factors may be determined in cell culture media using any suitable assay, such as an antibody-based assay (e.g., ELISA). The presence or level of an angiogenesis marker may also be determined by testing for the presence of one or more cell surface markers of angiogenesis, which method may also employ immunological methods, including ELISA. Further, commercial services exist, and may be used for determining levels of secreted factors, including the Beadlyte® and CytokineProfiler™ Testing Service, an ELISA-based assay offered by Millipore Corporation (Temecula, Calif.). Generally, the levels of markers associated with angiogenesis are compared to levels of one or more control markers that are not associated with angiogenesis. Such control markers are numerous, and are known in the art.

Alternatively, to determine the level of expression of angiogenesis-related factors, the level of cellular RNA (either total RNA, polyA+mRNA, or cDNA) may be analyzed using any platform for determining RNA expression levels, including DNA microarrays. The microarray may contain probes, not only for the angiogenesis-related factors, but also probes for nucleic acids that are characteristic of particular proliferative disease states, and/or genes associated with disease progression and drug resistance. Various microarrays are available from a number of commercial sources, such as Affymetrix, Incyte Pharmaceuticals, Stratagene, Nanogen and Rosetta lnpharmatics. The National Human Genome Research Institute (NHGRI) also has begun a collaborative research effort entitled “The Microarray Project,” which includes such efforts as the development of microarrays, robotic microarrayers and automated readers. DNA microarrays can include hundreds to many thousands of unique DNA samples covalently bound to a glass slide in a very small area. By hybridizing labeled RNA, mRNA, or cDNA to the array, the altered expression of one or more genes may be identified.

After hybridization with labeled cellular nucleic acids the relative amount of bound label at each discreet location of the microarray is determined. When labeled RNA or cDNA is hybridized to the microarray, the intensity of the label at each location of the microarray is generally directly proportional to the quantity of the corresponding mRNA species in the sample. Labeled cDNA or RNA from two cell types (i.e., normal and diseased proliferating cells) may be hybridized to the microarray to identify differences in RNA expression profiles for both test and control cells. Tools for automating microarray assays, such as robotic microarrayers and readers, are available commercially from companies, such as Nanogen, and are under development by the NHGRI. The automation of microarray analysis is desirable because of the large number of samples that may be interpreted.

Chemoresponse Testing

In addition to preparing an angiogenic signature, cells from the monolayer may be inoculated into at least one segregated test site for chemoresponse testing. In accordance with some embodiments, the invention provides a method for combining chemoresponse testing and evaluating angiogenic potential, without establishing separate cultures. For example, as disclosed herein, cultures maintained under normoxic conditions for chemoresponse testing are suitable for determining the presence and/or levels of several angiogenesis-related factors in a manner that sufficiently represents the in vivo hypoxic environment. Thus, cells may be seeded in a single plate (or single well) for both chemoresponse testing and for assaying secreted factors related to angiogenesis.

In these embodiments, the present invention may be used in connection with the proprietary ChemoFx® assays, which involve the isolation, short-term growth, and drug dosage treatment of epithelial cells derived from solid tumors. This assay is described below.

At the time of surgical “debulking,” or biopsy (e.g., vacuum-assisted and core biopsy) or fine needle aspiration of a tumor site, pieces of solid tumor are obtained by the surgeon, radiologist, or pathologist and placed in tissue culture media. The tumor is minced into small pieces and placed with cell culture media (Lifetech, Gibco BRL) into small flasks or other appropriately sized culture dishes for cell outgrowth. Over time, cells move out of the tumor pieces and form a monolayer on the bottom of the vessel. Once enough cells have migrated out of the ex vivo explant pieces, they are then trypsinized and reseeded into microtiter plates for either ChemoFx® Assay (versions 1 and 2 described below), for assay of cell culture media, or for immuno-histochemistry (IHC) analysis.

In Version 1 of the ChemoFx® Assay, cultured cells are seeded into 60 well microtiter plates at a density of about 100-500 cells per well and allowed to attach and grow for about 24 hours. After about 24 hours in culture the cells are then exposed for about 2 hours to a battery of chemotherapeutic agents. At the end of the incubation with the chemotherapeutic agents, the plates are washed to remove non-adherent cells. The remaining cells are fixed with 95% ethanol and stained with the DNA intercalating blue fluorescent dye, DAPI, or 6-diamidino 2-phylindole dihydrochloride (Molecular Probes, Eugene, Oreg., USA) or equivalent. The surviving cells are then counted using an operator-controlled, computer-assisted image analysis system (Zeiss Axiovision, Thornwood, N.Y., USA). A cytotoxic index is then calculated. The data are presented graphically as the cytotoxic index (CI). A dose-response curve is then generated for each drug or drug combination evaluated.

For the Version 2 ChemoFx® Assay, proprietary software, named Resource Allocator, is utilized to generate logical scripts that direct the activity of a liquid handling machine. The procedure, however, may be carried out using any liquid handling machine with appropriate software. This software employs the ideology behind the assay, a plating cell suspension of about 4,000 to 12,000 cells/ml and 1-10 replicates per dose for each of a multiple dose drug treatments, to calculate the number of cells necessary to accommodate testing of all requested drugs. In one embodiment, the assay comprises about 8,000 cells/ml and 3 replicates per dose for each of 10 dose drug treatments. After those calculations are complete, Resource Allocator will determine the quantity of disposable pipette tips, 8 row deep-well basins and 384 well microplates necessary for cell plating as well as the location of those consumables on the stage of the liquid handler. Finally, Resource Allocator will determine the specific location of cells in an 8 row deep-well basin prior to plating, and the specific location of cells in a 384 well microplate after plating. This information is provided in a printable format for easy interpretation of results. Using the information provided by Resource Allocator, a cell suspension is prepared at a concentration of about 4,000 to 12,000 cells/ml and delivered to a reservoir basin on the stage of the liquid handling machine. The machine then seeds about 200 to 400 cells in about 30 to 50 μl of medium into the wells of a 384 well microplate in replicates of about 1-10, after which the cells are allowed to adhere to the plate and grow for about 24 hours at 37° C. In one embodiment, the cell suspension is prepared at a concentration of about 8,000 cells/ml, and the liquid handling machine seeds about 320 cells in about 40 μl of medium into the wells of a microplate in replicates of 3.

After all cell suspensions have been delivered to the appropriate 384 well microplate, Resource Allocator is initiated again to calculate the number of drugs, and volume of each, that are needed to accommodate treatment of all cells plated. The software uses a volume of about 30-50 μl per replicate for each dose of a drug treatment and the number of unique cell lines needing that particular treatment to calculate the total volume of drug required. For instance, the software may use a volume of about 40 μl per replicate for each dose. After determining the necessary volume of each drug, the software calculates the number of disposable pipette tips, 96 well deep-well plates, and medium basins necessary for drug preparation. Resource Allocator will then determine into which 96 well deep-well plate each drug will go, the specific location in a 384 well microplate the treatment will be delivered, and the stage location for all of the consumables. For ease of interpretation, Resource Allocator provides these results in a printable format.

Following the approximately 4-28 hour incubation of the cell plates, the liquid handling machine prepares ten doses of each drug, in the appropriate growth medium, via serial dilutions in a 96 well deep-well microplate. When the drugs are ready, the liquid handling machine dispenses 30-50 μl of a drug (at 2× the final testing concentration) into the appropriate wells of the deep well plate. After treatment, the drugs can be left on the cells for an incubation of about 25-200 hours thus necessitating their preparation in growth medium. The drugs may be left on the cells for an incubation of 48-96 hours. During this period, cell viability is maintained with a standard incubator. During imaging of the cells, their viability is maintained with a device named the BioBox and visible light images are taken at predetermined intervals using proprietary software named Plate Scanner. The BioBox is a humidified incubator environment on the stage of a microscope. While the procedure uses the BioBox, other equipment known in the art may be used in practice. Temperature and gas composition are maintained at 37° C. and 5% CO₂ with air balance, respectively. It serves the purpose of providing an environment suitable for cell growth, while maintaining limited exposure to ambient air, which reduces potential contamination of the plates. Plate Scanner automates the acquisition of images from each well that has received cells in a microtiter plate. Plate Scanner provides the ability to choose which wavelengths of light to use as well as the ability to decide exposure duration for each wavelength of light chosen. in addition, the software uses focal stack imaging to determine the physical geometry of each plate in order to optimize image quality. The software automatically alters the light (either visible, UV or fluorescent) to capture the necessary image and stores the image on a hard drive. While the procedure uses Plate Scanner, other equipment and software known in the art may be used in practice.

At the end of the 25-200 hour incubation period, the liquid handling machine is used to remove the media and any non-adherent cells. Then, the remaining cells are fixed for at least 20 minutes in 95% ethanol followed by the DNA intercalating blue fluorescent dye, DAPI. Following fixation and staining, the automated microscope is used to take visible and UV images of the stained cells in every well. Afterwards, the number of cells per well in both visible and UV light is quantified using proprietary software named Cell Counter.

Cell Counter scans through each unique image and ascertains the cell locations by measuring the peak pixel intensity and aggregating pixels that are significantly above the background signal. The software provides various filters, such as minimum pixel intensity threshold, which allow better distinction of cells from background noise. While the procedure uses Cell Counter, any cell counting machine known in the art may be used in the practice of the methods of the inventions disclosed herein.

A complete dose response curve is generated for each drug evaluated. An Image analysis system is used in analysis of the cells. Here, cells grown in plates are imaged using equipment and methods known to those of ordinary skill in the art.

In the agent assays, growth of cells is monitored to ascertain the time to initiate the assay and to determine the growth rate of the cultured cells; sequence and timing of agent addition is also monitored and optimized. By subjecting uniform samples of cells to a wide variety of pharmaceutical agents (and concentrations thereof), the most efficacious agent or combination of agents can be determined.

A two-stage evaluation may be carried out in which both acute cytotoxic and longer term inhibitory effects of a given anti-cancer agent (or combination of agents) are investigated.

Predictive Models

In a second aspect, the invention provides a method for preparing a predictive model that finds use in predicting angiogenic and/or metastatic potential of a tumor, as well as predicting efficacy of anti-angiogenic therapy. In accordance with this aspect, the method comprises culturing malignant cells from a plurality of patient tumor specimens (e.g., using methods described herein), and preparing an angiogenic signature (as described herein), for each tumor specimen. The angiogenic signatures are then matched or correlated with treatment regimens and clinical outcomes for the patients from which the specimens originated. For example, the predictive model may correlate the signatures with the progression of disease and the outcome of treatment from the clinical record, which may include the results of treatment with: a monoclonal antibody against VEGF (e.g., Bevacizumab), soluble VEGF receptors (e.g., VEGF Trap), tyrosine kinase receptor inhibitors (e.g., inhibitors of VEGFR 1, 2, and/or 3, FLT3, PDGFR-α and/or β) inhibitors of endothelial proliferation (e.g., Endostatin, Angiostatin, Thalidomide), inhibitors of extracellular matrix breakdown (e.g., Marimastat, Neovstat), and inhibitors of vascular adhesion (e.g., Vitaxin).

An angiogenic signature prepared for a patient's tumor specimen is matched with the closest representative signature(s) in the predictive model, to evaluate the angiogenic potential of the tumor in vivo (e.g., based on disease progression in the clinical record), and/or to determine whether a particular anti-angiogenic therapy might be effective (e.g., based upon the clinical record). In some embodiments, computer algorithms are used for carrying out pattern matching between the test signature and model signatures. A linear regression algorithm, for example, can be used to analyze a database and identify the signature that most closely matches the signature for the patient's tumor. In one embodiment, a comparative analysis of signatures is performed using a known linear regression algorithm.

The presence or levels, or relative levels, of angiogenic markers may be incorporated into an algorithm to predict a response to anti-angiogenesis agents. For instance, an increase or decrease in concentration of one or more angiogenesis-related factors may be readily observed. An algorithm of patient outcome versus analyte levels can be trained and tested in a multivariate analysis to predict how a patient will respond to a particular anti-angiogenic therapy. Such an algorithm can incorporate data for two or more, three or more, four or more or five or more angiogenesis-related factors. The data may be analyzed with or without the aid of a computer. Computers employing a regression or other algorithm, including, but not limited to, SVM, Decision Tree, LDA and PCA may be employed.

Additional Markers for Providing Further Predictive Value

The predictive value of the invention may be enhanced by combining the information regarding the presence or levels of angiogenesis-related factors secreted from tumor cells in vitro, with the presence or levels of additional factors, either present in the cell culture or present in a biological sample taken from the patient (such as a blood, saliva, or urine sample).

Nucleic acids isolated from the patient's cells may be analyzed to identify markers that are characteristic of abnormally proliferating cells, or associated with disease progression, and which may add additional predictive value when choosing a therapeutic regimen. For example, the method of the invention may further comprise determining the presence in the patient's tumor cells of one or more tumor suppressor genes, oncogenes, translocations, and/or mutations associated with cancer, or resistance to chemotherapy.

The method of the invention may include testing for the presence or levels of various markers associated with the progression of disease and/or metastasis (e.g., in a biological sample from the patient). Such markers include urokinase or an MMP, such as MMP2, MMP7, and/or MMP9. Further phenotypic analysis, such as for cell adhesion, migration, chemotaxis and invasion, can offer additional predictive value. In one embodiment, the method also tests for endogenous inhibitors of angiogenesis, such as platelet factor 4, angiostatin, and endostatin, in a biological sample from the patient. These embodiments lend additional predictive value regarding the angiogenic state of the tumor.

A number of substances secreted by tumor cells, such as tumor associated antigens and plasminogen activators and inhibitors, are believed to regulate a variety of processes involved in the progression of malignant disease. Many of these factors are produced by tumor cells growing in tissue culture and are secreted into the growth medium. The measurement of these factors in the medium from cell cultures of tumor specimens may also prove to be of predictive value in the assessment of the biological behavior of individual cancers. For example, culture medium may also be assayed for the presence or absence of secreted tumor antigens, such as PAI-1, u-PA, cancer associated serum antigen (CASA) or carcinoembryonic antigen (CEA). These factors may be detected through use of standard assays, such as radioimmunoassay (RIA) or enzyme-linked immunosorbent assay (ELISA), or other antibody-based assay.

The cell cultures may also be assayed histochemically and/or immunohistochemically for identification or quantification of cellular or membrane-bound markers. Examples of such markers include CEA, tissue polypeptide specific antigen TPS, EGFR, TGFB receptor and mucin antigens, such as CA 15-3, CA 549, CA 27.29 and MCA. Markers indicative of complications of a proliferative disease may also be analyzed. For instance, one common complication is thrombogenesis. A propensity towards blood clot formation can be detected in tissue culture medium by identifying thrombogenic or procoagulant factors such as, without limitation, cancer cell-derived coagulating activity-1 (CCA-1), the Lewis Y antigen (Ley), HLA-DR and other tumor procoagulants, such as cancer procoagulant (CP) and tissue factor (TF). By identifying production of thrombogenic factors, a physician can prescribe drug and/or exercise regimens, as appropriate, to prevent life and/or limb-threatening clotting.

### Examples

### Production of Angiogenic Factors Under Hypoxic Conditions

Based on physiological in vivo conditions, it was hypothesized that cells grown in a hypoxic in vitro environment will express angiogenic-inducing factors at higher levels than those grown under normoxic conditions. Those factors associated with VEGF production are expected to increase in response to the hypoxic environment, while IP-10 should decrease. A secondary goal was to determine whether primary tumors exhibit differential expression of angiogenic-related factors.

Primary cell cultures were established using tumor specimens procured for research purposes from the following sources: National Disease Research Interchange (NDRI) (Philadelphia, Pa.), Cooperative Human Tissue Network (CHTN) (Philadelphia, Pa.), Forbes Regional Hospital (Monroeville, Pa.), Jameson Hospital (New Castle, Pa.), Saint Barnabas Medical Center (Livingston, N.J.), Hamot Medical Center (Erie, Pa.), and Windber Research Institute (Windber, Pa.). Upon receipt, all specimens were minced to a fine consistency with Cincinnati Surgical #10 or #11 scalpels (PGC Scientifics, Frederick, Md.), followed by antibiotic washes, as necessary. In order to establish primary cultures, the specimens were typically divided into 25 cm² and/or 75 cm² Cellstar® sterile tissue culture flasks with filtered caps (PGC Scientifics, Frederick, Md.), depending on the desired seeding density. Cell culture media were tumor type specific: breast tumors were cultured in Mammary Epithelial Growth Media (MEGM; Lonza Bio Science Walkersville, Walkersville, Md.), ovarian tumors were cultured in McCoy's 5A growth media (Mediatech, Herndon, Va.), lung tumors were cultured in Bronchial Epithelial Growth Media (BEGM; Lonza Bio Science Walkersville), and colon tumors were cultured in RPMI 1640 growth media (Mediatech). The amount of Fetal Bovine Serum (FBS; HyClone, Logan, UT) present in the media was also tumor-type specific, as was the presence of PureCol™ collagen (Inamed Biomaterials, Fremont, Calif.) on the culture surface. Antibiotic washes and antibiotic media were formulated with Penicillin-Streptomycin Solution (Mediatech), Gibco Gentamicin Reagent Solution (Invitrogen Corporation, Grand Island, N.Y.), Fungisone (Invitrogen), Cipro® I.V. (ciprofloxacin) (Oncology Therapeutics Network, South San Francisco, Calif.), and Nystatin (Sigma-Aldrich, St. Louis, Mo.). Other reagents include Trypsin EDTA (0.25%) and Hanks Buffered Saline Solution with and without Calcium and Magnesium (HBSS) (Mediatech).

All cultures were initially established in humidified incubators at 37° C. with 5% CO₂ for 5 to 28 days. When a confluency of at least 30 percent was attained, cells were trypsinized, counted, and plated as described below.

Three human tumor-derived immortalized cell lines were also tested: SK-OV-3, ovarian adenocarcinoma; MDA-MB-231, mammary adenocarcinoma; and A549, lung carcinoma (American Type Culture Collection, Manassas, Va.). These cell lines were seeded at 50,000 cells per 5 ml in T25 flasks and allowed to grow for one week to approximately 90% confluency. At that time, the cells were trypsinized, counted, and plated as described below.

After the initial culture period, a total of fifty samples (45 primary cultures and 5 cell line samples) were trypsinized, counted, and suspended in culture media to a concentration of 40,000 cells/ml. Each of the samples was plated at 20,000 cells/well into one well of two separate Greiner 24-well culture plates (CLP Molecular Biology, San Diego, Calif.). Both plates were maintained under normoxic conditions (5% CO₂ and 21% O₂) for 48 hours to allow for cell adherence and equilibration. After 48 hours, one plate remained in normoxic conditions while the other plate was transferred to a NAPCO Series 8000WJ Water Jacketed CO₂ Incubator (ThermoFisher Scientific, Waltham, Mass.) where hypoxic conditions were established. Nitrogen gas was injected to purge the incubator of oxygen resulting in a final O₂ concentration of 1% while the CO₂ concentration was maintained at 5% (Mukherjee et al., 2005). Plates were incubated for an additional 48 hours. At the end of the incubation period, the confluency for each sample was recorded and the supernatant was collected and stored at −80° C.

Collected supernatants were sent to Millipore Corporation (Temecula, Calif.) for protein evaluation via the Beadlyte® CytokineProfiler™ Testing Service, an ELISA-based assay. Evaluated angiogenesis-related cytokines and growth factors included: VEGF, PDGF-AA, PDGF-AA/BB, IL-8, bFGF, EGF, IP-10, Flt-3 ligand, TGF-β1, TGF-β2, and TGF-β3. Additionally, RANTES, an analyte not related to angiogenesis, was tested as a negative control for a subset of samples. For each analyte, two replicates were performed using 40 μl of supernatant per replicate.

For each analyte, protein expression levels in the normoxic and hypoxic conditions of all samples were combined into an x-y scatter plot. Then, a linear regression of the curve fit for protein concentration under the hypoxic versus normoxic condition was generated for each analyte tested. For all linear regressions, y=mx+b, y is the concentration produced in the hypoxic environment and x is the concentration produced in the normoxic condition. From this regression, the slope, intercept, and correlation of determination (r²) were calculated. The strength of each linear relationship was determined by the r² value of the linear regression, with r² values greater than 0.8 considered strong relationships, and r² values between 0.6 and 0.8 considered moderate relationships. The same parameters were used to assess VEGF expression levels by tumor type. Lastly, comparisons were generated between the eleven angiogenesis-related factors studied for every cell source. The difference between the protein expression level under the hypoxic condition versus the normoxic condition was calculated. This value was standardized on a scale of zero to one, with zero set equal to the lowest value observed and one set equal to the highest value observed. These values were then graphed as a heat map for all samples across all factors. Correlation coefficients were determined for each factor in relation to VEGF expression.

### Results

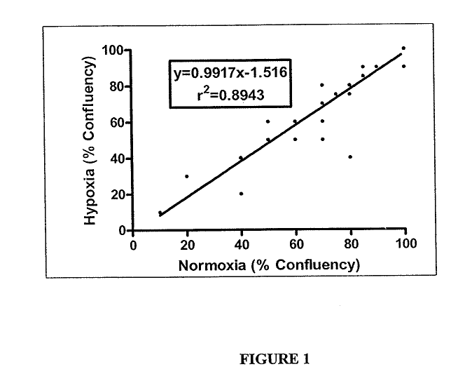

The study included fifty distinct cell populations. Forty-five primary tumor specimens were designated based on final pathology and site of tumor origin including: 10 breast, 15 lung, 13 ovary, 3 colon, 3 central nervous system (CNS), and 1 unknown primary. Additionally, five cell line samples were tested including: A549, one sample; MDA-MB-231, one sample; and SK-OV-3, three samples. All samples were evaluated under both normoxic and hypoxic environments in parallel. A strong linear relationship for the confluency of the normoxic versus hypoxic condition existed across all samples, with a linear regression of y=0.9917x−1.516 (r²=0.8943; FIG. 1).

Hypoxia-Induced Expression of Angiogenesis-Related Factors

Moderate to strong linear relationships of the protein expression levels between hypoxic and normoxic conditions were observed in eight of the eleven angiogenesis-related factors analyzed (Table 2). The strongest linear relationships (r²>0.95) are evident for IL-8, with hypoxic expression levels generally higher than normoxic (m=0.9627, b=569.1), and PDGF-AA, with lower levels in hypoxia (m=0.8322, b=−1.859). Strong correlations (r²>0.80) existed for a number of growth factors (all expressing similar levels under hypoxia and normoxia conditions), including: EGF (m=0.9497, b=−70); TGF-β2 (m=0.9632, b=22.65); and PDGF-AA/BB (m=1.015, b=3.74). One anti-angiogenic factor, IP-10, also had a strong linear correlation, with hypoxic expression levels lower than normoxic (m=0.8778, b=−27.55). Moderate correlations (r²>0.60) were observed for VEGF, with higher levels in hypoxia (m=1.174, b=552.2), and TGF-β1 (m=0.6186, b=194.7), with lower levels in hypoxia than normoxia. Correlations did not exist for bFGF or TGF-β3 (r²<0.25). Data for Flt-3 ligand was not evaluable, as only six of 50 samples had evaluable results.

TABLE 2
Linear correlations between normoxic and hypoxic growth conditions of
angiogenesis-related factors.

| | | Slope | | y-intercept | 95% CI y- | |
|---|---|---|---|---|---|---|
| Analyte | n | (m) | 95% CI Slope (m) | (b) | intercept | r² |
| VEGF | 50 | 1.174 | 0.9049 to 1.443 | 552.2 | 98.99 to 1005 | 0.6163 |
| bFGF | 27 | 0.0813 | −0.06828 to 0.2309 | 82.38 | 50.21 to 114.5 | 0.0478 |
| IL-8 | 33 | 0.9627 | 0.9076 to 1.018 | 569.1 | 2.366 to 1136 | 0.9761 |
| EGF | 22 | 0.9497 | 0.8266 to 1.073 | −70 | −357.1 to 217.1 | 0.9283 |
| PDGF-AA | 48 | 0.8322 | 0.7925 to 0.8720 | −1.859 | −20.92 to 17.21 | 0.9748 |
| PDGF-AA/BB | 21 | 1.015 | 0.8348 to 1.196 | 3.74 | −102.7 to 110.1 | 0.8793 |
| IP-10 | 35 | 0.8778 | 0.7738 to 0.9817 | −27.55 | −292.3 to 237.1 | 0.8995 |
| TGF-β1 | 45 | 0.6186 | 0.4914 to 0.7458 | 194.7 | 93.33 to 296.1 | 0.6913 |
| TGF-β2 | 47 | 0.9632 | 0.8808 to 1.045 | 22.65 | −226.8 to 272.1 | 0.9251 |
| TGF-β3 | 27 | 0.2433 | −0.1484 to 0.6350 | 23.36 | 10.64 to 36.07 | 0.0615 |

Hypoxia-Induced Expression of VEGF is Tissue-Type Dependent

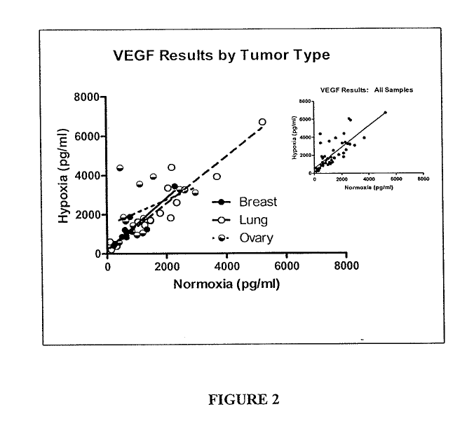

For VEGF, 46 of 50 samples exhibited higher expression levels in the hypoxic condition than in the normoxic condition. Since VEGF is the angiogenesis-related factor specifically implicated in the mechanism of action of bevacizumab, this data was further analyzed by tissue type (FIG. 2, Table 3). Overall, the combined results of all cell sources analyzed had a moderate correlation (r²>0.60). Breast, lung, and ovarian tumor types had sufficient sample sizes to sub-analyze by tumor type. While strong linear correlations were observed for breast and lung samples (r²>0.80), a linear correlation between hypoxic and normoxic expression of VEGF in ovarian samples did not exist (r²<0.25). Correlations were not available for CNS, colon and unknown primary tumors or for the cell lines, as samples sizes were too low to assess linearity.

TABLE 3
Linear correlations of VEGF between normoxic and hypoxic conditions.

| | | Slope | | y-intercept | 95% CI y- | |
|---|---|---|---|---|---|---|
| VEGF Results | n | (m) | 95% CI Slope | (b) | intercept | r² |
| Breast | 10 | 1.316 | 0.8360 to 1.795 | 206.1 | −262.5 to 674.7 | 0.8334 |
| Lung | 15 | 1.193 | 0.9280 to 1.458 | 178.1 | −422.0 to 778.3 | 0.8793 |
| Ovary | 13 | 0.6432 | −0.3679 to 1.654 | 1458 | 58.40 to 2858 | 0.1513 |
| All Samples | 50 | 1.174 | 0.9049 to 1.443 | 552.2 | 98.99 to 1005 | 0.6163 |

Differential Expression of Angiogenesis-Related Factors Across Patient Samples

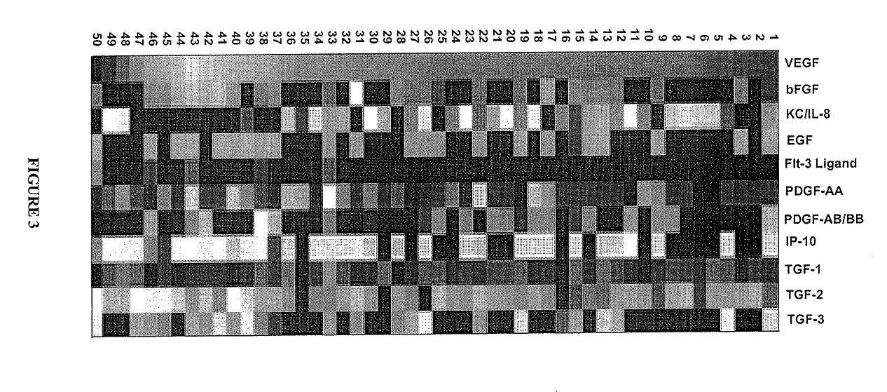

A heat-map of the differences between hypoxic and normoxic expression indicates expression levels of angiogenesis-related factors differed both within and between patients (FIG. 3). This data was specifically sorted by VEGF expression from lowest to highest difference for a visual representation of the heterogeneous expression levels. Correlation coefficients were also calculated for all nine angiogenesis-related factors with evaluable data in relationship to VEGF (data not shown). No correlation was greater than 0.5, indicating differences in the other angiogenesis-related factors are not correlated to differences in VEGF expression. Together, these data reinforce the idea that differential angiogenesis-related protein expression levels exist for each sample.

### Discussion of Results

This example addresses a number of topics related to the expression of angiogenesis-related factors in normoxic versus hypoxic environments. Specifically, (1) linear correlations exist for a number of angiogenesis-related factors, (2) linear correlations for VEGF exist and group by tumor type, and (3) primary expression levels vary between samples and across factors.

Linear correlations between protein expression in normoxic and hypoxic environments exist for eight of the eleven angiogenesis-related factors tested in this study (Table 2). Hypoxic expression levels were generally higher than normoxic for IL-8 (r²>0.95) and VEGF (r²>0.60). IL-8 regulates angiogenesis by promoting survival of endothelial cells, stimulating matrix metalloproteinases, and increasing endothelial permeability (Cheng et al., 2008; Petreaca et al., 2007). VEGF is a major signaling protein for angiogenesis secreted in higher levels when cells experience hypoxia (Muller et al., 1997). Both of these factors are expressed to induce vascular growth due to hypoxia in vivo, and appear to do the same in vitro. IP-10, an anti-angiogenic factor, had lower expression levels in the hypoxic condition than in the normoxic condition (r²>0.80). This was expected, as this protein inhibits tumor growth by regulating lymphocyte chemotaxis and inhibiting endothelial growth (Sato et al., 2007).

Trends in the expression levels of other growth factors were variable. Lower expression levels were observed in the hypoxic condition for PDGF-AA (r²>0.95) and similar levels were observed for PDGF-AA/BB (r²>0.80). These results are not surprising as platelet populations are minimal in culture. These cells are non-adherent to flask surfaces and are rinsed away during routine media changes. Different results were observed for each of the transforming growth factors, likely related to the specific role each plays in cancer pathogenesis (Mourskaia et al., 2007). Lower expression levels were observed in the hypoxic condition for TGF-β1, while similar expression levels were observed in both conditions for TGF-β2 (r²>0.80) and no correlation existed for TGF-β3 (r²<0.25). Similar expression levels were observed in both conditions for EGF, which may be due to the fact that EGF induces VEGF, IL-8, and bFGF release by tumor cells, and is transformed in the process (Hicklin et al., 2005). A correlation did not exist for bFGF, which mediates VEGF production and induces extracellular matrix formation. Another in vitro study showed bFGF was unaffected by hypoxia in cell lines (Mukherjee et al., 2005). In all, the trends in protein expression levels observed suggest the hypoxic condition induced in vitro is similar to the change induced by tumor growth in vivo. Furthermore, the correlations between the conditions in vitro suggest the expression levels may be linked to in vivo expression of each angiogenesis-related factor, whether measured in normoxic or hypoxic conditions.

The combined results of all cell sources analyzed for VEGF showed a moderate correlation between normoxic and hypoxic expression levels. Stronger linear correlations were observed for breast and lung samples specifically. Breast and lung samples are cultured in unique culture media as compared to ovarian, CNS, and colon samples. Primary breast tumors are cultured in Mammary Epithelial Growth Media (MEGM), while lung tumors are cultured in Bronchial Epithelial Growth Media (BEGM). These media require addition of SingleQuots® to basal media that include EGF. Significantly, EGF induces VEGF, IL-8, and bFGF release by tumor cells (Hicklin et al., 2005). While this SingleQuots® may have contributed to the VEGF production in these tumor types, the other analytes (IL-8 and bFGF) induced by EGF did not correlate by tumor type (data not shown). Therefore, culture media is probably not responsible for the differential expression levels of the ten evaluable angiogenesis-related proteins and a unique fingerprint for each sample. In general, these data suggest in vitro expression levels of VEGF can be measured in either a normoxic or a hypoxic condition, since a linear correlation exists between expression levels in both conditions.

Differential protein expression levels existed for each factor tested in this study, as is evident in FIG. 3. In vitro studies show differential degrees of primary tumor response to chemotherapy agents in vitro. Furthermore, these response rates correlate with progression-free interval in ovarian cancer patients, which indicates in vitro tests performed on primary cultures may be used to enhance the probability of choosing the best treatment regimen for the patient (Gallion et al., 2006, Int J Gynecol. Cancer 16: 194-201). Similarly, differential protein expression levels existed across patients in this study for each of the factors. This supports the concept of a predictor for angiogenesis-related anticancer agents using an array of protein expression levels observed in vitro. While toxicity, delivery, metabolism and clearance affect patient response to therapeutics in vivo, in vitro studies are commonly used in initial testing of novel treatments and have clinical potential when applied to primary cultures (Kornblith et al., 2004, Int J Gynecol Cancer 14: 607-615).

Although extreme hypoxic conditions may compromise the health of the cells and lead to cell death, similar confluencies between the normoxic and hypoxic condition at the conclusion of testing prove that the 48 hour incubation prior to testing was sufficient for cell adherence and equilibration (Table 1). To support this observation, Pilch et al. found that hypoxia did not induce a decrease in cell culture confluencies (Pilch et al., 2001).

Also, in addition to the 11 angiogenesis-related analytes chosen for testing, a negative control unrelated to angiogenesis was also assessed. A chemotactic cytokine, Regulated upon Activation, Normal T-cell Expressed, and Secreted (RANTES), is responsible for recruiting leukocytes and activating natural killer cells (Maghazachi et al., 1996, Eur J Immunol 26(2): 315-319). This cytokine was not expected to vary in a normoxic versus hypoxic environment. Results for six samples were available and indicate similar expression levels for both conditions, with a linear regression of y=1.0411x+0.0807 and r²=0.9924.

Multiple techniques are available to assess VEGF expression. Some laboratories employ immunohistochemical (IHC) analysis to determine VEGF receptor levels (Mohammed et al., 2007), usually for diagnostic and prognostic purposes. However, this study employed the Beadlyte® CytokineProfiler™ Testing Service for two reasons. First, this service provides quantitative analysis of the expression levels of the angiogenesis-related factors, including VEGF. Second, testing was performed on epithelial cell cultures. When IHC is used, tissue sections are generally stained at tissue extraction and VEGF receptors on endothelial cells and monocytes fluoresce. However, neither of these cell types is present in these samples because the culture process selects specifically for malignant epithelial cells (Heinzman et al., 2007, Pathology 39(5): 491-494). Endothelial cells are selected against by culture conditions, as the media employed do not promote the growth of these cells. Monocytes are non-adherent, so are rinsed away in routine media changes.

VEGF production was of most interest to this study due to its role in the mechanism of action of bevacizumab. The testing conditions were optimized to ensure that VEGF production was measurable, so VEGF results were available for all samples tested. Table 3 includes the summary of all data in the “All Samples” field, a total of 50 samples. Results for the other ten analytes had detection levels out of range of the standard curve for at least two samples, if not more. As a result, the sample size for most of these angiogenesis-related factors was less than 50 (Table 2). However, nine of these ten factors had at least 20 samples available for analysis, and were considered evaluable in the study.

As with any anticancer therapeutic agent, there is clinical ambiguity regarding individual patient response. Some agents directly target VEGF, such as bevacizumab, a humanized monoclonal antibody, while others indirectly target receptors and downstream regulators, such as sunitinib and rituximab (Wang et al., 2004). The protein expression levels produced by individual patient cells may provide information on how each patient will respond clinically to a given anticancer agent. The heterogeneity of protein expression demonstrated in this study may provide information to enable the prediction of the efficacy of anti-angiogenic factors. Further studies correlating the in vitro expression levels with patient outcome are warranted.

### Conclusions

Linear correlations exist between expression levels of angiogenesis-related factors under normoxic and hypoxic conditions. This suggests the behaviour of primary cells derived from patient tumors grown under in vitro normoxic conditions may provide a correlation to the in vivo hypoxic environment. Differential expression for each sample across all factors suggests predictive value for angiogenesis-related anti-cancer agents, using not only VEGF, but an array of angiogenesis-related proteins.

The present invention has been described with reference to specific details of particular embodiments thereof. It is not intended that such details be regarded as limitations upon the scope of the invention except insofar as and to the extent that they are included in the accompanying claims. All patents and publications cited are herein incorporated in their entireties for all purposes.

1. A method for creating an angiogenic signature for a tumor specimen, comprising: culturing malignant cells from a patient tumor specimen; and testing the cell culture for the presence and/or levels of at least three angiogenesis- related factors selected from VEGF/VPF, bFGF/FGF-2, IL-8/CXCL8, EGF, Flt-3 ligand, PDGF-AA, PDGF-AA/BB, IP-10/CXCL10, TGF-β1, TGF-β2, and TGF-β3.

2. The method of claim 1 , wherein the cell culture is maintained under normoxic conditions.

3. The method of claim 1, wherein cell cultures are maintained in hypoxic and normoxic environments in sequence or in parallel, and the presence and/or levels of the angiogenesis-related factors are tested in both.

4. The method of claim 1, wherein at least five of VEGF, PDGF-AA, PDGF-AA/BB, IL-8, bFGF/FGF-2, EGF, IP-10/CXCL10, Flt-3 ligand, TGF-β1, TGF-β2, and TGF-β3 are tested.

5. The method of claim 1, wherein all of VEGF, PDGF-AA, PDGF-AA/BB, IL-8, bFGF/FGF-2, EGF, IP-10/CXCL10, Flt-3 ligand, TGF-β1, TGF-β2, and TGF-β3 are tested.

6. The method of claim 1, wherein the tumor specimen is a breast or lung cancer.

7. The method of claim 1, wherein the cell culture is enriched for malignant cells.

8. The method of claim 7, wherein the malignant cells are cultured from cohesive multicellular particulates of the patient tumor specimen.

9. The method of claim 1, wherein the cells from the cell culture are further exposed to chemotherapeutic agents for chemoresponse testing.

10. A method for preparing a predictive model for predicting efficacy of angiogenic therapy, comprising:
culturing malignant cells from a plurality of patient tumor specimens;
preparing an angiogenic signature for each tumor specimen by testing each cell culture for the presence and/or level of at least three angiogenesis-related factors selected from VEGF/VPF, bFGF/FGF-2, IL-8/CXCL8, EGF, Flt-3 ligand, PDGF-AA, PDGF-AA/BB, IP-10/CXCL10, TGF-β1, TGF-β2, and TGF-β3; and
matching the angiogenic signatures with treatment regimens and clinical outcomes for said patients, to thereby prepare a predictive model.

11. The method of claim 10, wherein the cell culture is maintained under normoxic conditions.

12. The method of claim 10, wherein cultures are maintained in hypoxic and normoxic environments in sequence or in parallel, and the presence and/or levels of the angiogenesis-related factors are tested in both.

13. The method of claim 10, wherein the angiogenic signature comprises the test results for at least five of VEGF, PDGF-AA, PDGF-AA/BB, IL-8, bFGF/FGF-2, EGF, IP-10/CXCL10, Flt-3 ligand, TGF-β1, TGF-β2, and TGF-β3.

14. The method of claim 10, wherein the aniogenic signature comprises the test results for all of VEGF, PDGF-AA, PDGF-AA/BB, IL-8, bFGF/FGF-2, EGF, IP-10/CXCL10, Flt-3 ligand, TGF-β1, TGF-β2, and TGF-β3.

15. The method of claim 10, wherein the tumor specimens are breast and/or lung cancer specimens.

16. The method of claim 10, wherein the cell cultures are enriched for malignant cells.

17. The method of claim 16, wherein the malignant cells are cultured from cohesive multicellular particulates of a patient tumor specimen.

18. The method of claim 10, wherein the patient treatment regimens include treatment with bevacizumab.

19. The method of claim 10, wherein the patient outcomes include resistance or development of resistance to said anti-angiogenic treatment.

20. A method for predicting efficacy of an anti-angiogenic treatment, comprising:
culturing malignant cells from a patient tumor specimen;
preparing an angiogenic signature for the tumor specimen by testing the cell culture for the presence and/or levels of at least three angiogenesis-related factors selected from VEGF/VPF, bFGF/FGF-2, IL-8/CXCL8, EGF, Flt-3 ligand, PDGF-AA, PDGF-AA/BB, IP-10/CXCL10, TGF-β1, TGF-β2, and TGF-β3; and
matching the angiogenic signature with a model signature correlated to angiogenic treatment regimen and patient outcome.

21. The method of claim 20, wherein the cell culture is maintained under normoxic conditions.

22. The method of claim 20, wherein cultures are maintained in hypoxic and normoxic environments in sequence or in parallel, and the presence and/or levels of the angiogenesis-related factors are tested in both.

23. The method of claim 20, wherein at least five of VEGF, PDGF-AA, PDGF-AA/BB, IL-8, bFGF/FGF-2, EGF, IP-10/CXCL10, Flt-3 ligand, TGF-[beta]1, TGF-[beta]2, and TGF-[beta]3 are tested.

24. The method of claim 20, wherein all of VEGF, PDGF-AA, PDGF- AA/BB, IL-8, bFGF/FGF-2, EGF, IP-10/CXCL10, Flt-3 ligand, TGF-[beta]1, TGF-[beta]2, and TGF-[beta]3 are tested.

25. The method of claim 20, wherein the tumor specimen is a breast or lung cancer.

26. The method of claim 20, wherein the cell culture is enriched for malignant cells.

27. The method of claim 26, wherein the malignant cells are cultured from cohesive multicellular particulates of a patient tumor specimen.

28. The method of claim 20, wherein the cells from the cell culture are exposed to chemotherapeutic agents for chemoresponse testing.
